(19) United States
(12) Patent Application Publication
(10) Pub. No.: US 2026/0030756 A1
(43) Pub. Date: Jan. 29, 2026

(54) Disease Relevance Determination Program and Disease Relevance Determination Apparatus

(71) Applicants: TAHESHI HASHIMOTO, NAGOYA-SHI, AICHI (JP); LOCAL INCORPORATED ADMINISTRATIVE AGENCY TOKYO METROPOLITAN INSTITUTE OF GERONTOLOGY, TOKYO (JP)

(72) Inventors: MASASHI KAMEYAMA, TOKYO (JP); TOMOMICHI IIZUKA, TOKYO (JP); YUMI KAMEYAMA, TOKYO (JP); MASAHIRO AKISITA, TOKYO (JP)

(73) Assignees: TAHESHI HASHIMOTO, NAGOYA-SHI, AICHI (JP); LOCAL INCORPORATED ADMINISTRATIVE AGENCY TOKYO METROPOLITAN INSTITUTE OF GERONTOLOGY, TOKYO (JP)

(21) Appl. No.: 18/851,629

(22) PCT Filed: Mar. 27, 2023
(86) PCT No.: PCT/JP2023/012303

§ 371 Date: Mar. 31, 2025

(30) Foreign Application Priority Data
Mar. 28, 2022 (JP) 2022-051137

(51) Int. Cl. G06T 7/00; G16H 70/60

(52) U.S. Cl. CPC G06T 7/0014; G16H 70/60; G06T 2207/20081; G06T 2207/20084; G06T 2207/30004; G06T 2207/30201

(57) ABSTRACT

[Problem] To provide: a dementia determination device which enables the accurate determination of dementia in a non-invasive, inexpensive and simple manner; and a dementia determination program.

[Solution] Provided is a disease relevance determination program for making a computer execute the below-mentioned steps to determine the disease relevance in a test subject, in which the steps include: a feature point acquisition step for acquiring weighed feature point data X in a reference subject using default face photo data of a smiley face of the reference subject which have been acquired in advance; a distinction data acquisition step for acquiring data A correspo DISTINCTION [symbol] he weighed feature point from the face photo data for DISTINCTION [symbol] ject; and a determination step for comparing the data A with the data X to determine the disease relevance in the test subject. Also provided is a disease relevance determination program which is provided with a computer in which the disease relevance determination program is loaded.

### TECHNICAL FIELD

The present invention relates to a disease relevance (this means whether or not it corresponds to a disease) determination apparatus and a disease relevance determination program capable of non-invasively, inexpensively, easily, and accurately performing disease relevance determination such as dementia determination.

### BACKGROUND ART

There are various methods for disease relevance determination such as dementia diagnosis, and amyloid positron emission tomography (PET), cerebrospinal fluid biomarkers, and the like have been proposed. However, the amyloid PET is very expensive and has an invasive disadvantage of radiation exposure. The cerebrospinal fluid biomarkers are highly invasive because insertion of a needle into the spinal cavity is required. Therefore, a non-invasive, inexpensive, and simple method for assisting dementia diagnosis is desired, and various proposals have been made.

For example, PTL 1 proposes a dementia test system with improved determination accuracy. Specifically, the dementia test system proposed in PTL 1 includes an information delivery unit that delivers information to a test subject by exciting at least one of a visual sense or an auditory sense of the test subject, a measurement unit that measures a response of the test subject to which the information is delivered, and a control unit that calculates a plurality of different feature amounts on the basis of a measurement result of the measurement unit and determines whether or not the test subject has dementia on the basis of the feature amounts. The measurement unit includes a sound collection unit that collects a voice uttered by the test subject, and the control unit determines whether or not the test subject has dementia at least on the basis of at least one acoustic feature amount that is an acoustic feature amount of the voice uttered by the test subject and at least one language feature amount that is a linguistic feature amount of the voice uttered by the test subject.

PTL 2 proposes a physiological state determination apparatus and a physiological state determination method for easily determining a physiological state of a test subject. Specifically, the physiological state determination apparatus proposed in PTL 2 includes a facial change information acquisition unit, a facial change information decomposition unit, and a physiological state determination unit. The facial change information acquisition unit acquires facial change information indicating a time-series change in face data of the test subject. The facial change information decomposition unit decomposes the facial change information into a plurality of components by singular value decomposition, principal component analysis, or independent component analysis. The physiological state determination unit determines a mental or physical physiological state of the test subject based on a determination component extracted from the plurality of components.

PTL 3 proposes a dementia diagnosis apparatus capable of achieving a highly accurate dementia diagnosis without giving a patient a psychological resistance. Specifically, there is proposed an apparatus including: a speech acquisition unit that acquires speech data related to a conversation between a test subject and an interrogator; a speech analysis unit that performs speech analysis on the speech data to specify a type of a question content in an utterance section, in which the interrogator makes an utterance, and to extract an answer feature in an utterance section, in which the test subject makes an utterance, following the utterance section; and a dementia level calculation unit that inputs the answer feature of the test subject in association with the type of the question content to a trained identifier and calculates a dementia level of the test subject, wherein the identifier is subjected to learning processing so as to output the dementia level according to a predetermined dementia level determination rule when the answer feature of the test subject is input in association with the type of the question content.

### CITATION LIST

### Patent Literature

[PTL 1] Japanese Patent Application Laid-open No. 2018-15139.

[PTL 2] Japanese Patent Application Laid-open No. 2017-153938.

[PTL 3] Japanese Patent Application Laid-open No. 2019-84249.

### Non Patent Literature

[NPL 1] The Meeting of Japan Society for Dementia Research, Conflict-of-Interest Disclosure, Nov. 8, 2019, titled “Study on Early Detection of Dementia Using Deep Learning on Face Photographs”.

[NPL 2] Umeda-Kameyama Y, Kameyama M, Tanaka T, Son BK, Kojima T, Fukasawa M, Iizuka T, Ogawa S, Iijima K, Akishita M. Screening of Alzheimer's disease by facial complexion using artificial intelligence. Aging (Albany NY). 2021 Jan. 25; 13(2): 1765-1772. Doi: 10.18632/aging.202545. Epub 2021 Jan. 25.

### SUMMARY OF INVENTION

### Technical Problem

However, the apparatus or the like according to the

above proposals has not been able to determine dementia with sufficient accuracy yet.

In order to solve these problems of the proposals, the present inventors proposed, in NPL 1 and NPL 2, a method capable of easily determining dementia by causing artificial intelligence (AI) to perform determination on a face, but there has been a demand for development of a more accurate determination method.

Therefore, an objective of the present invention is to provide a disease relevance determination apparatus and a disease relevance determination program capable of non-invasively, inexpensively, easily, and accurately determining dementia.

### Solution to Problem

As a result of intensive studies to solve the above problems, the present inventors have found that the above object can be achieved by using not only mere face data but also two of a normal face and a smiling face as face data, and have completed the present invention.

That is, the present invention provides the following inventions.
1. A disease relevance determination program for causing a computer to perform steps below to determine disease relevance in a test subject, the steps including:
a feature point acquisition step of acquiring data X corresponding to a weighted feature point in a reference subject known to have the disease by using default smiling face photograph data acquired from the reference subject prior to the feature point acquisition step;
a distinction data acquisition step of acquiring data A corresponding to a weighted feature point from face photograph data of the test subject; and
a determination step of determining disease relevance in the test subject by comparing the data A with the data X.
2. The program according to 1, wherein in the determination step, the determination is performed based on whether or not a specific value of the data A is 0.5 or more.
3. The program according to 1, wherein the feature point relates to a state of eyes and a mouth.
4. The program according to 1, wherein the weighted feature point is based on a state of eyes in the smiling face photograph data.
5. A disease relevance determination apparatus including:
one or more computers that store the disease relevance determination program according to 1;
an input means for inputting predetermined data to the one or more computers; and
an output device that outputs a determination result stored in the one or more computers.
6. The disease relevance determination apparatus according to 5, wherein the one or more computers that perform the distinction data acquisition step has an additional function comprising a question-and-answer function for having the test subject to have a conversation with the one or more computers or an image display function for showing an image to the test subject.

### Advantageous Effects of Invention

The disease relevance determination apparatus according to the present invention can non-invasively, inexpensively, easily, and accurately perform disease relevance determination such as dementia determination. In addition, the disease relevance determination program according to the present invention can provide the disease relevance determination apparatus according to the present invention.

### BRIEF DESCRIPTION OF DRAWINGS

FIG. 1 is an explanatory diagram schematically illustrating an overall configuration of a dementia determination apparatus serving as a disease relevance determination apparatus according to the present invention.

FIG. 2 is a flowchart schematically illustrating a flow of a dementia determination program serving as a disease relevance determination program according to the present invention.

FIG. 3 illustrates a sheet (including a photograph used) obtained by visualizing a learning process with gradient-weighted class activation mapping (Grad-CAM) in an embodiment of the disease relevance determination program according to the present invention.

### REFERENCE SIGNS LIST

1 Dementia determination apparatus
10 Computer
11 Memory
13 CPU
15 Storage medium

### DESCRIPTION OF EMBODIMENTS

Hereinafter, preferred embodiments of the present invention will be described in detail with reference to the drawings.

In the following description, dementia will be described as an example of a “disease”. In the present invention, the “disease” is not limited to dementia, and examples of the “disease” include various diseases that affect the face, such as Parkinson's disease, Lewy body dementia, and depression.

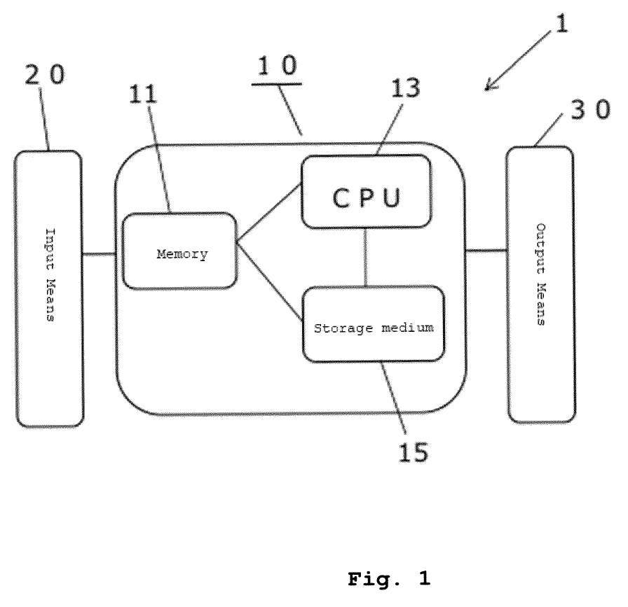

As illustrated in FIG. 1, a dementia determination apparatus 1 serving as a disease relevance determination apparatus according to the present embodiment includes: one or a plurality of computers 10 that store a dementia determination program according to the present embodiment described below; input means 20 for inputting predetermined data to the computer; and output means 30 for outputting a determination result of the computer.

### Computer

Specifically, as illustrated in FIG. 1, the computer 10 used in the present embodiment includes a central processing unit (CPU) 13, a memory 11 serving as a temporary storage area, and a nonvolatile storage medium 15 such as a hard disk or a solid state device. In the present invention, as the “computer”, in addition to a normal personal computer, a server or a mobile terminal such as a so-called smartphone or a tablet terminal can also be used. Further, a computer for performing a feature point acquisition step and a computer for performing other steps may be provided separately for the program described below. For example, the computer for performing the feature point acquisition step may be a personal computer or a server, and the computer for performing other steps may be a tablet. It is a matter of course that one computer may be caused to perform all of the steps of the program according to the present invention described below, so that all of the steps can be performed.

Although not particularly illustrated, the computer in the present embodiment preferably includes a communication device and can perform communication via a network. The computer can also be connected to a server including a database and provided on a network and set to obtain updated data from the database as needed by performing communication.

In addition, although not particularly illustrated, a graphics processing unit (GPU) is also preferably mounted on the computer according to the present embodiment, particularly, the computer for performing the feature point acquisition step.

In the present embodiment, the program according to the present embodiment described below is stored in a storage medium 15 of the computer, and the computer is caused to function as means for determining dementia.

### Input Means

Although not particularly illustrated, an image input device such as a keyboard, a mouse, or a camera, a speech input device such as a microphone, a communication input device using a communication device such as Bluetooth (registered trademark), or the like is used as the input means 20. Accordingly, necessary data and information can be input if appropriate.

In the present embodiment, a camera is particularly important input means, and data accumulation and determination are performed using a face photograph of a test subject captured by the camera.

### Output Means

A display for displaying an evaluation result, a printer for printing, or the like is used as the output means 30. Such output means outputs the determination result in a desired form, and the determination result is used by, for example, the test subject and a user such as a doctor who utilizes data.

### Addition of Other Functions

Preferably, the dementia determination apparatus 1 according to the present embodiment includes at least a speech input device and an image input device serving as the input means, and includes at least a display and a speaker for speech output as the output means. Then, the computer is preferably configured such that the program described below causes the computer to perform a smiling face creation step of adding a question-and-answer function for causing the test subject to have a conversation with the computer or a video display function for showing a video to the test subject. The smiling face creation step is described below.

Here, both the question-and-answer function and the video display function are for causing the test subject to make a smiling face. A definition of the smiling face is described below.

The question-and-answer function is a function of presenting a question to the test subject by the output means and asking the test subject to answer to create an environment in which the test subject easily smiles and acquire a natural smiling face. The question to be prepared may be an individual specific question for each test subject or a general question.

In addition, the video display function is a function of displaying, by the output means, a relaxing video or a video that is likely to cause a smile to acquire a natural smiling face of the test subject.

### Other Members (Devices)

The apparatus according to the present embodiment can include various devices as necessary in addition to the above-described devices.

### Program

The program according to the present embodiment is a program that is stored in the computer and causes the computer to perform the following steps to determine whether or not the test subject has dementia.

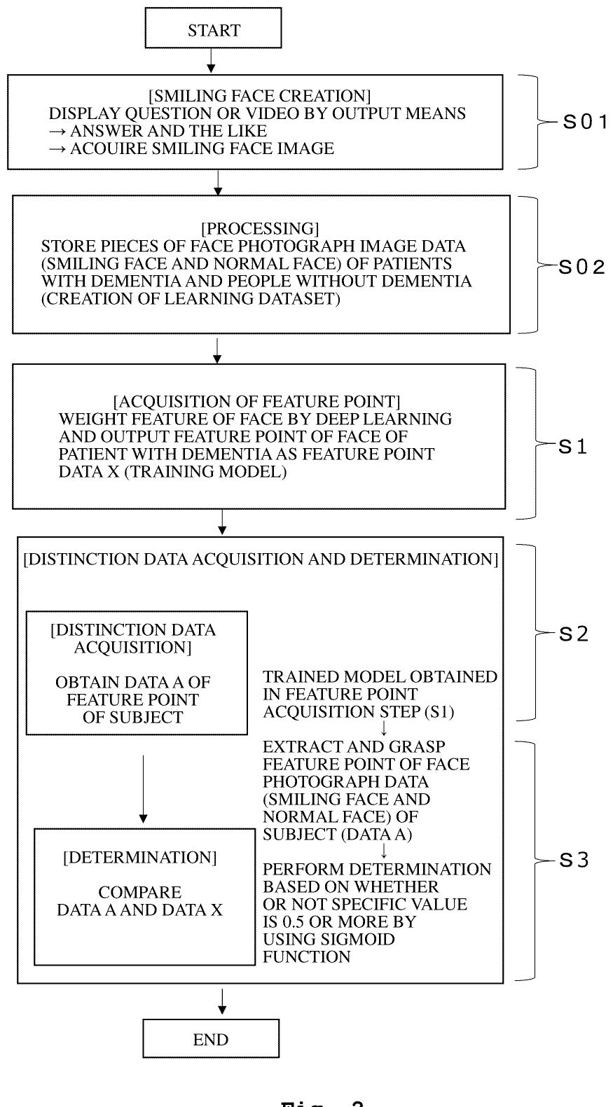

As illustrated in FIG. 2, the steps include:

a feature point acquisition step (S1) of acquiring data X of a weighted feature point in a reference subject known to have dementia by using default face photograph data acquired in advance;

a distinction data acquisition step (S2) of acquiring data A corresponding to the weighted feature point from the face photograph data of the test subject; and

a determination step (S3) of determining whether or not the test subject has dementia by comparing the data A with the data X.

### Preprocessing Step (S02)

In the program according to the present embodiment, first, the default face photograph data is acquired and stored in the storage medium.

Here, a normal face photograph and a smiling face photograph are acquired as the face photograph data. The smiling face photograph means a photograph taken with an instruction of “smile” at the time of photographing, and the normal face photograph means a photograph taken with no instructions or simply with a standard prompt that is not a special instruction, such as “I'm going to take a picture”, at the time of photographing. As described below, instead of the instruction “smile”, a moving image or a speech may be provided to the test subject such that the test subject naturally smiles, thereby acquiring the smiling face photograph. In addition, it is preferable to acquire, as the face photograph data, the normal face photograph and the smiling face photograph, which are captured from the front and from a diagonal forward angle. In addition, the “standard prompt that is not a special instruction” includes a prompt for taking a photograph without smiling, such as “maintain a poker face”, “do not smile”, or “as if for an identification photograph”.

The smiling face includes a smiling face made by the test subject himself/herself according to the instruction to make a smiling face and a natural smiling face made by listening to a conversation or watching a video, which can be suitably used in the present invention. Since some subjects have difficulty in making an artificial smiling face, a natural smiling face can be acquired by using a smiling face creation step described below.

Furthermore, in a case where a learning model is obtained by deep learning described below, it is preferable to store the face photograph data as a learning dataset with a dementia patient tag and other tags attached thereto (see FIG. 2).

### Feature Point Acquisition Step (S1)

The feature point acquisition step is a step of acquiring the data X of the weighted feature point in a reference subject known to have dementia by using the default face photograph data acquired in the preprocessing step.

Here, the feature point relates to a state of the eyes and the mouth, and in particular, it is preferable to use the eyes as the weighted feature point in the smiling face photograph data, and further, it is preferable to use the eyes as the weighted feature point in the smiling face photograph data and use the mouth as the weighted feature point in the normal face photograph data.

That is, in the present embodiment, smiling face data and normal face data are discriminated from the face photograph data, data of the eyes is grasped as the feature point in the smiling face data in the discriminated data, and data of the mouth is grasped as the feature point in the normal face data.

The feature point is grasped and extracted by identifying how the movements of the eyes and the mouth differ between a dementia patient and people without dementia to grasp a face state peculiar to a dementia patient (detection of the smiling face).

In the present embodiment, the grasping and extraction operation can be performed by deep learning. Specifically, Keras, with TensorFlow (registered trademark) (which is a software library developed by Google Inc., and “TENSORFLOW” is a registered trademark) as a backend, can be used. Then, learning can be performed by a system (for example, VGG-Face or exception) capable of machine learning of a face image. In this case, it is preferable to perform machine learning by using four datasets of smiling face photographs or normal face photographs. Specifically, it is preferable to use a smiling face photograph and a normal face photograph of a patient with dementia (hereinafter, referred to as “AD”) and a smiling face photograph and a normal face photograph of a patient with normal cognition (hereinafter, referred to as “NC”) to use four datasets of a combination of the smiling face photograph and the normal face photograph in the case of NC (Dataset 1), a combination of the smiling face photograph and the normal face photograph in the case of AD (Dataset 2), the smiling face photographs in the case of both of AD and NC (Dataset 3), and the normal face photographs in the case of both of AD and NC (Dataset 4).

A configuration of a neural network used in the deep learning according to the present embodiment is similar to a known technology. That is, the neural network outputs a feature point of the face of a patient with dementia from an output layer by sequentially propagating (calculating) information input to an input layer to an intermediate layer and the output layer. For example, the intermediate layer includes a plurality of intermediate units. Then, the information input to an input unit of the input layer is weighted (integrated) by each coupling coefficient (not illustrated) and input to each intermediate unit of the intermediate layer, and the pieces of information are added to be a value of each intermediate unit. The value of each intermediate unit of the intermediate layer is nonlinearly converted by an input/output function (for example, a sigmoid function), weighted (integrated) by each coupling coefficient (not illustrated), and input to an output unit of the output layer, and the values are added to become a value (the feature point of the face) of the output unit of the output layer. More preferably, as a convolutional neural network (also referred to as “CNN”), a neural network that includes a convolution layer, an activation or ReLU layer, a pooling layer, and the like and is directly trained using image data can also be used.

That is, a face photograph (smiling face and normal face) and data regarding whether or not the test subject has dementia are input to the input layer by the input means and transferred to the computer. The transferred data is then weighted in the intermediate layer by the plurality of intermediate units. Finally, in the output layer, data (trained model) of the feature points of the eyes and the mouth, which are the feature points in the face, is calculated as a weighted value.

In the present embodiment, it has been found that the feature point is at the eyes in the case of the smiling face and the feature point is at the mouth in the case of the normal face by the deep learning. That is, in a patient with dementia, a characteristic difference in the eyes is more conspicuous in the smiling face as compared with people without dementia, and thus the feature point in the smiling face is at the eyes, and a characteristic difference in the mouth is more conspicuous in the normal face, and thus the feature point in the normal face is at the mouth.

### Distinction Data Acquisition Step (S2)

The distinction data acquisition step (S2) is a step of acquiring the data A corresponding to the weighted feature point from the face photograph data of the test subject.

In this step, the data can be obtained by simply processing data of the eyes in the smiling face and data of the mouth in the normal face in the face photograph data of the test subject in association with the feature point data X grasped in the feature point acquisition step (S1).

This step can also be performed by performing training by a machine learning system such as the VGG-Face described above, but since the feature point has already been grasped by the feature point acquisition step (S1), desired data can be obtained in this step by performing processing in association with the feature point data obtained by the feature point acquisition step (S1).

In the present embodiment, this step can be performed integrally with the determination step (S3) described below.

### Determination Step (S3)

The determination step is a step of determining whether or not the test subject has dementia by comparing the data A with the data X. In the present embodiment, the determination is performed based on whether or not a specific value of the data A is 0.5 or more.

In the present embodiment, it is preferable to continuously perform the distinction data acquisition step (S2) and the determination step (S3). That is, in a case where the determination for the test subject is performed by deep learning, the feature point of the face photograph data (the smiling face and the normal face) of the test subject is extracted and grasped by using the trained model obtained in the feature point acquisition step (S1). At this time, when the face photograph data of the test subject is output by deep learning, it is determined whether or not the specific value at this time is 0.5 or more by using a sigmoid function f(x)=1/1+e^{-x}. For example, in a case where the specific value is less than 0.5, it is determined that a possibility of dementia is low, and in a case where the specific value is 0.5 or more, it is determined that the possibility of dementia is high.

For example, in a case where the above-described distinction data acquisition step (S2) is performed using the VGG-Face, the determination can be performed by group-based 10-fold cross validation. A learning curve can be created for 200 epochs. The possibility of dementia can be determined by the sigmoid function after determining the optimum number of epochs in consideration of the accuracy/loss and stability of each model and appropriately calculating diagnosis/prediction accuracy of the convolutional neural network (CNN) model.

### Other Steps

In the present invention, the program may be configured to cause the computer to perform the following steps in addition to the above steps.

### (Output Step (Not Illustrated))

An output step of outputting the determination result to the above-described output means 30 may be included.

An output form is not particularly limited, and the determination result may be output to the display or may be output in the form of a printout.

### (Smiling Face Creation Step (S01))

There may be included the smiling face creation step in which the dementia determination apparatus 1 according to the present embodiment adds, to the computer 10, the question-and-answer function for causing the test subject to have a conversation with the computer or a video display function for showing a video to the test subject.

In the smiling face creation step, speech data such as a question item and a conversation item, and video data such as various photographs and moving images are stored in advance in a recording medium, and the speech data and the video data are displayed by the output means such as a speaker or a display, and input is performed by the test subject by the input means such as a microphone, a keyboard, or a touch panel, so that the test subject is guided through an experience based on the speech and/or the video, encouraging the test subject to make a natural smiling face. A state of the test subject at this time is photographed by the camera (input means), and an image of the smiling face is acquired.

Here, the question item and the video are not particularly limited as long as the question item and the video can make a person naturally smile.

### Implementation Method/Effect

The disease relevance determination program and the disease relevance determination apparatus according to the present invention can be used as follows.

That is, the disease relevance (dementia relevance) to the test subject can be determined by executing the above-described disease relevance determination program using the above-described dementia determination apparatus 1.

Specifically, a disease relevance determination method can be performed to determine the disease relevance to the test subject by performing a step of acquiring data of the smiling face and the normal face in advance, the feature point acquisition step of acquiring the data X of the weighted feature point in a reference subject known to have the disease from the acquired data of the faces, the data acquisition step of acquiring the data A that is determination data from data of a face photograph of the test subject, and the determination step of comparing the data X and the data A and determining the disease relevance to the test subject.

According to the disease relevance determination program and the disease relevance determination apparatus of the present embodiment, it is possible to determine disease (dementia or the like) relevance to the test subject in the steps as described above. Since it is possible to discriminate whether or not the test subject has the disease only by preparing or acquiring two types of face photographs of the normal face and the smiling face, it is possible to simply and easily determine the disease. In addition, since no special test is required, a burden on the test subject is small, and even in the case of a disease that causes significant resistance from the test subject, such as dementia, determination can be performed without causing discomfort of the test subject, which is useful for early detection of the disease.

The present invention is not limited to the above-described embodiments, and various modifications can be made without departing from the gist of the present invention.

For example, the present invention is also applicable to diseases (for example, Parkinson's disease, Lewy body dementia, and depression) that affect emotions and facial expressions other than dementia.

In addition, it is preferable to perform deep learning from the feature point acquisition step to the determination step, but in the feature point acquisition step, a threshold value for determination may be extracted, and the data A acquired in the distinction data acquisition step may be compared with the threshold value in the determination step to perform determination.

Furthermore, in the above-described embodiment, a case where the feature point is acquired from the data of the smiling face has been described as an example. However, it is also possible to acquire the feature point from a face other than the smiling face, that is, the normal face, acquire the feature point by combining the feature point of the normal face with the data of the smiling face, acquire the determination data, and perform the determination.

Hereinafter, the present invention will be described in more detail with reference to Examples, but the present invention is not limited thereto.

### Example 1

A trained model was created for 280 patients with dementia (hereinafter, referred to as “AD”) and 190 patients with normal cognition (hereinafter, referred to as “NC”) at the Medical Center for Dementia Diseases in FUKUJUJI hospital by using a computer storing the program described in the above-described embodiment [the preprocessing step (S02) and the smiling face creation step (S01)].

The relevance to the AD was diagnosed based on criteria of the National Institute of Neurological and Communicative

Disorders and Stroke and the Alzheimer's Disease and Related Disorders Association (NINCDS-ADRDA). A Hachinski ischemic scale of each patient with AD was ≤4. Normal face photographs and smiling face photographs of all subjects were captured from the front and from a diagonal forward angle.

### Feature Point Acquisition Step (S1)

The following four datasets were created for binary differentiation:

A combination of the smiling face photograph and the normal face photograph in the case of NC (Dataset 1), a combination of the smiling face photograph and the normal face photograph in the case of AD (Dataset 2), the smiling face photographs in the case of both AD and NC (Dataset 3), and the normal face photographs in the case of both AD and NC (Dataset 4).

An approach based on transfer learning was applied for the detection of the smiling face.

A network was constructed using an open-source neural network library Keras with a symbolic tensor manipulation framework TensorFlow (registered trademark) (Google, Mountain View, CA, USA) as the backend, and the Adam optimizer.

Detection was performed by training using the VGG-Face. A VGG16 trained in advance with 2.6 million face images by the VGG-Face (a deep neural network developed by Visual Geometry Group, which is trained so as to be specialized for face images) includes five convolution (Conv) blocks, each of which includes two to three Conv layers and pooling layers (hereinafter, the VGG-Face may be referred to as “VGG-FaceCNN”). The first Conv block has two Conv layers and a MaxPooling layer in a cascade manner. Outputs of the layers were inputs of the second Conv block. For example, it is assumed that a first Conv layer of the first block receives an input of 224×224×3 for a color image of 224×224. After successive convolution and pooling operations on different blocks, a size of an output of the VGG-16 model is 7×7×512. The size is further converted into a linear vector of 7×7×512, an output of 25,088 is output as a vector of 1,000 outputs by linear operation, and the vector is input to the last layer including 128 outputs. The final output for seven emotion expressions based on the inputs and outputs was 7. Settings and parameters of the Adam as the optimizer were a learning rate 0.00001, β1 =0.9, β2 =0.999, =None, and decay=0.0, amsgrad=False. Training image data was reinforced under the following conditions: rotation range: 15, height shift range: 0.03, width shift range: 0.03, shear range: 5, zoom range: 0.1, horizontal inversion: true, vertical inversion: false, luminance range: 0.3 to 1.0, and channel shift range: 5.

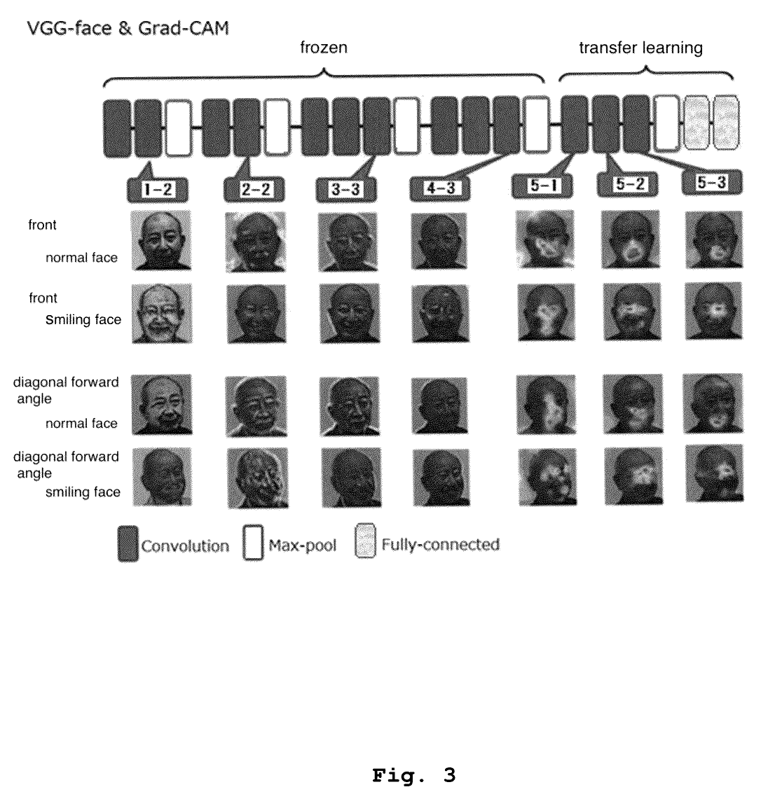

The first four Conv blocks of the VGG-Face were frozen as illustrated in FIG. 3 for fine tuning. A Conv block 5 was then trained using the above four datasets of the smiling face photographs or the normal face photographs. As a result, the data X, which is the feature point data, was acquired.

### Distinction Data Acquisition Step (S2) and Determination Step (S3)

The acquisition of the data A, which is data for determination by the VGG-Face, and determination were substituted by performing group-based 10-fold cross validation without specially preparing a test subject. The learning curve was prepared for 200 epochs. The optimum number of epochs was determined in consideration of the accuracy/loss and stability of each model, and the diagnosis/prediction accuracy of the convolutional neural network (CNN) model was calculated by the group-based 10-fold cross validation.

The amount of information obtained by applying an inverse sigmoid function to an output prediction value by a combination of four data subsets and the VGG-FaceCNN as in this example was as follows.

A binary differentiation of the smiling face and the neutral expression of the face image (Dataset 1) is described as “smiling face/neutral/NC score”, and a differentiation of the smiling face of the face images of AD and NC (Dataset 3) is described as “smiling face/AD/NC score”. In addition, a binary differentiation (dataset 4) of the neutral expression in the AD face image and the NC face image is expressed as a “neutral-AD/NC score”. The scores were obtained by applying the inverse sigmoid function to the output prediction value. Sensitivity, specificity, accuracy, and area under the receiver operating characteristic (ROC) curve (AUC) were calculated for the scores by using the 10-fold cross validation.

A proportion of the smiling face photographs identified as the smiling faces was 91.6% in the case of NC (Dataset 1) and 67.1% in the case of AD (Dataset 2), suggesting that it is difficult for a significant portion of the AD group to make a smiling face. Thereafter, when a smiling face/normal face-NC score was applied to the AD group with the smiling faces, 48.6% was classified as negative. It can be seen from this result that it is possible to determine dementia with high probability by checking whether image data of a smiling face is recognized as a smiling face.

Another visual determination was made, and 69.5% of the smiling face photographs of the NC group were visually determined as smiling faces of people with Duchenne disease. An average smiling face/neutral/NC score of the smiling faces was as high as 3.89 (SD, 0.59). An average positive smile/neutral-NC score of smiling faces of people without Duchenne disease in this group was 2.13 (SD, 0.98). A difference in this score was significant (p=0.00035).

Further, gradient weighted class activation mapping (Grad-CAM) was applied to define which part of the face the VGG-FaceCNN identifies. The VGG-Face is a VGG16 trained in advance with 2.6 million face photographs. The first four blocks were frozen and five blocks were trained with a dataset prepared for transfer learning, and the process was visualized with the Grad-CAM. The results are illustrated in FIG. 3. The CNN roughly captures a contour of the face in the first block, and focuses on constituent elements of the face in the second and subsequent blocks. The CNN focused mainly on both eyes and the mouth in Conv 5-1 in the fifth block, and one of both eyes and mouth was selected in Conv 5-3. The Grad-CAM mainly focuses on the mouth and the corner of the mouth of the normal face in the case of AD and NC. In the smiling face in the case of AD and NC, particularly when a smiling face/normal face/NC score was positive, heat maps were frequently arranged on the eyes, the outer corners of the eyes, and the eyebrows. Therefore, it can be seen that the determination is made focusing on the mouth in the expression of the normal face and on the eyes in the expression of the smiling face.

It can be seen from the above results that, by applying the program according to the present invention, it is possible to determine dementia with high probability only by acquiring a face photograph.

1. A disease relevance determination program for causing a computer to perform steps below to determine disease relevance in a test subject, the steps including:
a feature point acquisition step of acquiring data X corresponding to a weighted feature point in a reference subject known to have the disease by using default smiling face photograph data acquired from the reference subject prior to the feature point acquisition step;
a distinction data acquisition step of acquiring data A corresponding to a weighted feature point from face photograph data of the test subject; and
a determination step of determining disease relevance in the test subject by comparing the data A with the data X.

2. The program according to claim 1, wherein in the determination step, the determination is performed based on whether or not a specific value of the data A is 0.5 or more.

3. The program according to claim 1, wherein the feature point relates to a state of eyes and a mouth.

4. The program according to claim 1, wherein the weighted feature point is based on a state of eyes in the smiling face photograph data.

5. A disease relevance determination apparatus including:
one or more computers that store the disease relevance determination program according to claim 1;
an input means for inputting predetermined data to the one or more computers; and
an output device that outputs a determination result stored in the one or more computers.

6. The disease relevance determination apparatus according to claim 5, wherein the one or more computers that perform the distinction data acquisition step has an additional function comprising a question-and-answer function for having the test subject to have a conversation with the one or more computers or an image display function for showing an image to the test subject.
